(19) United States
(12) Patent Application Publication
(10) Pub. No.: US 2011/0223209 A1
(43) Pub. Date: Sep. 15, 2011

(54) Oxygen delivering scaffold for tissue engineering

(76) Inventors: Roel Kuijer, Groningen (NL); Sjoerd Klaas Bulstra, Groningen (NL); Albert Gerrit Veldhuizen, Eelde (NL); Honglian Dai, Wuhan (CN)

(21) Appl. No.: 13/055,919

(22) PCT Filed: Jul. 25, 2008
(86) PCT No.: PCT/NL08/50518

§ 371 Date: May 17, 2011

(51) Int. Cl. A61K 9/00; C12N 5/071; A61K 35/12; A61K 35/28; A61K 35/32; A61K 33/40; A61P 43/00; A61P 19/08; B29B 11/16

(52) U.S. Cl. 424/400; 435/395; 424/93.7; 424/93.21; 435/325; 435/377; 424/613; 264/294

(57) ABSTRACT

The invention relates to compositions, implantable devices and methods related to tissue engineering. Provided is a composition comprising (i) at least one biocompatible polymer suitable for use in tissue-engineering scaffolds and (ii) at least one metal peroxide. Also provided is an implantable device comprising said composition, and methods for tissue engineering comprising the use of the device.

The invention relates to compositions, implantable devices and methods related to tissue engineering.

Tissue engineering (TE) aims to restore, maintain, and/or improve tissue function(s). The US National Science Foundation defines tissue engineering as “the application of principles and methods of engineering and life sciences to obtain a fundamental understanding of structure-function relationships in novel and pathological mammalian tissues and the development of biological substitutes to restore, maintain, or improve tissue function.” In the last decade, over $3.5 billion dollars has been invested worldwide in TE research.

Typically, TE involves the seeding of autologous primary cells from the tissue which they intend to develop on a biocompatible and preferably degradable scaffold, followed by culturing the cell-scaffold construct under conditions favouring the development of the intended tissue. Alternatively, stem cells can be used which can differentiate in the required lineage given the appropriate conditions. Using these approaches most tissues can be produced in an immature form.

Several aspects of creating an engineered tissue make it a daunting task. One challenge is concerned with directing the behaviour of specialized cells outside of the body to mimic the normal, endogenous phenotype those cells exhibit in vivo. Biomaterial scaffolds are fundamental components of many tissue engineering strategies. The scaffold can serve as both a physical support and adhesive substrate for cells during in vitro culturing and subsequent implantation in vivo. Scaffolds can be utilized to deliver cells to desired sites in the body, to define a potential space for engineered tissue, and/or to guide the process of tissue development. Cell transplantation on scaffolds has been explored for the regeneration of, among others, skin, heart, nerve, liver, pancreas, cartilage, and bone tissue using various biological and synthetic materials.

In order for an engineered tissue to be tolerated upon implantation, the material that provides the scaffolding for the cells must meet several important criteria. The material must be biocompatible, so as not to be toxic or injurious, and not cause immunological rejection. Because cells respond biologically to the substrate on which they adhere, the materials that provide the growth surface for engineered tissues must promote or at least support cell growth. Additionally, the material should allow cells to grow and function as they would in vivo.

One of current limitations of tissue engineering is the inability to provide sufficient blood supply in the initial phase after implantation. Almost all cells in the mammalian body are highly dependent on oxygen, the only exception possibly being chondrocytes, which originate from a non-vascularised tissue, cartilage, and are adapted to survive in low-oxygen conditions. Oxygen is normally delivered through erythrocytes, which take up oxygen in the lungs and distribute the oxygen during their journey through the vascular network in exchange for carbon dioxide. A proper vascularisation is essential for the vitality of our tissues.

In normal cell culture conditions, the oxygen concentration is relatively high as compared to the oxygen concentration in the body. In addition, the culture medium contains all required nutrients for cell growth and differentiation. Thus, the establishment of a cell-scaffold construct in vitro is in general not limited by the supply of oxygen and/or nutrients. The problem arises when such cell (tissue)-scaffolds are implanted in a tissue lesion. Then the cells (tissue) are suddenly exposed to an environment with both a very low oxygen concentration and nihil nutrients. The wound healing reaction, which always occurs after surgical intervention, provides a surrounding with a variety of cytokines and growth factors, aiming at closing the wound as soon as possible, and is accompanied by an inflammatory reaction with activated granulocytes and later macrophages.

Vascularisation does take place, but at a slow rate. It often takes several days to weeks until scaffolds of 1 cm thick are completely vascularised. As a consequence, oxygen-dependent cells will die due to the ischemia, in a necrotic way exposing the cell content, which will further strengthen the inflammatory reaction.

The above problem is well recognized in the art, and several efforts are being undertaken to improve the vascularisation of TE-scaffolds. One strategy involves providing tube-like structures to scaffolds which should be populated by endothelial cells and connected to the host vascular system, or by mixing either primary cells or stem cells with endothelial cells. Other approaches to maintaining tissue viability of engineered tissue include the use of oxygen-rich fluids, such as perfluorocarbons and silicone oils, and the use of angiogenic factors. However, despite these attempts the clinical applicability of engineered tissues has been hampered by limited tissue survival, in particular of oxygen-sensitive tissue and/or large tissue mass.

The present inventors therefore set out to provide a novel approach to increase the supply of oxygen to transplanted tissues. It was surprisingly found that this goal was met by seeding cells on a scaffold comprising at least one metal peroxide. Upon contact with an aqueous environment, the metal peroxide releases oxygen in situ for a prolonged period of time, thereby ensuring an increased oxygen supply to the cells or tissue.

Accordingly, the invention relates to a composition comprising (i) at least one biocompatible polymer suitable for use in tissue-engineering scaffolds and (ii) at least one metal peroxide. The metal ion of the metal peroxide is preferably a divalent, biocompatible cation. Suitable biocompatible metal peroxides include zinc peroxide (ZnO₂) calcium peroxide (CaO₂), magnesium peroxide (MgO₂) and mixed calcium/magnesium peroxide (Ca,MgO₂). Preferred metal peroxides are CaO₂ and MgO₂. Oxygen release starts upon contact with water. For a divalent metal (Me) peroxide, the reaction is as follows:

2MeO₂+2H₂O [symbol] 2Me(OH)₂+O₂

Oxygen release from CaO₂ was found to be a relatively quick process, lasting for approximately 4-7 days. In contrast, oxygen release from MgO₂ is a slow process, lasting for at least several weeks. Accordingly, a combination of CaO₂ and MgO₂ is advantageously used to obtain an oxygen-delivering scaffold capable of providing oxygen in situ with the desired kinetics, e.g. not only immediately upon implantation but also for a prolonged period of time.

Harrison et al. (Biomaterials 28 (2007), 4628-4634) discloses an implantable oxygen releasing biomaterial consisting of sodium percarbonate incorporated into a film of PLGA. Oxygen release was observed over a period of hours, and the reaction was complete after 24 hours. The use of metal peroxides in tissue engineering as disclosed herein is not suggested, let alone their capacity to provide cells or tissues with oxygen over a period up to several weeks.

In one aspect, a composition comprises CaO₂ and MgO₂ in a relative amount of between 10:1 and 1:10 by weight, more preferably between 5:1 and 1:5. In one embodiment, about equal amounts of both oxygen sources are used. In another embodiment, the slow releasing MgO₂ is present in excess of CaO₂. For example, the relative weight ratio between CaO₂ and MgO₂ ranges from between 1:1.1 to about 1:10, preferably from about 1:2 to about 1:8, such as 1:3, 1:4 or 1:5. In yet another embodiment, the fast releasing CaO₂ is present in excess of MgO₂. For example, the relative weight ratio between CaO₂ and MgO₂ ranges from between 1.1:1 to about 10:1, preferably from about 2:1 to about 8:1, such as 3:1, 4:1 or 5:1.

The total amount of oxygen-delivering metal peroxide in a composition of the invention can vary, again depending on the desired oxygen-delivering characteristics. The total amount of metal peroxide(s) will generally be at least 2 weight %, preferably at least 5 weight %, more preferably at least 10 weight % of metal peroxide based on the total dry weight of the composition. In this context it is to be noted that oxygen can be very toxic to cells and tissue at high concentrations. In addition, as is described herein below, oxygen release by peroxides is accompanied with the formation of hydroxides which cause a (local) increase in pH. Therefore, very high (initial) levels of oxygen release are preferably avoided. In one embodiment, the amount of metal peroxides does not exceed 20 weight %, preferably 15 weight %. For instance, the metal peroxides are present in an amount of 2-20 weight %, preferably 5-20 weight %, such as 5-17 weight %, 5-15 weight %, like 7 weight %, 10 weight %, 12 weight %, 14 weight %.

As will be appreciated by the skilled person, the concept underlying the present invention can in principle be applied to any existing or yet to be discovered type of material that is suitable for use in tissue engineering. A material or composition that can be used as a scaffold in tissue engineering must satisfy a number of requirements. These include biocompatibility, processability to complicated shapes with appropriate porosity, ability to support cell growth and proliferation, appropriate mechanical properties as well as maintaining mechanical strength during the tissue regeneration process. To ensure sufficient mechanical strength, the polymer content of a composition according to the invention will typically be at least 60 weight %, preferably at least 70 weight %, more preferably at least 80 weight % based on the total dry weight of the composition. In one embodiment, the polymer content ranges from between 80 and 98 weight %, such as 82, 85, 87, 90, 92, 95, 96 or 97 wt %.

As said, any type of biocompatible polymer used in the art of tissue engineering can be used. The term ‘biocompatible’ is used to describe materials that are non-toxic to the host organism. Thus, biocompatible materials do not compromise the function of the host organism. During the initial seeding of cells into/on the scaffold it is important that the materials used for the production of the three dimensional scaffold are biocompatible. But also during the degradation of scaffolds or, alternatively, during long term in situ placement of the scaffold it is important that toxic degradation products do not occur or if they do occur the release of toxic compounds is sufficiently slow to avoid the building up of toxic compounds.

The polymer in a composition of the invention can be biodegradable or non-biodegradable. Biodegradable polymers can be attractive for use in scaffolding compositions because they degrade as the new tissue is formed, eventually leaving nothing foreign to the body. The polymer can be natural or of synthetic origin. In one preferred embodiment, the polymer(s) in a composition as provided herein is/are synthetic (biodegradable) polymers. In contrast to natural polymers, synthetic polymers can be dissolved in an organic solvent. This allows to prepare a homogeneous suspension of MeO₂ particles in a solution of the polymer. By removing the solvent, the suspended particles are coated by the polymer and embedded in the polymer. Of course, exposure of metal peroxide with an aqueous solvent during scaffold manufacture should be avoided because this will cause premature and unwanted oxygen release. In a specific aspect, a composition comprises one or more synthetic biodegradable polymer(s). Synthetic biodegradable polymers for use in tissue engineering are well known in the art, see for example the review by Gunatillake et al. (European Cells and Materials, Vol. 5, 2003 (pages 1-16) and references cited therein. See also ‘Principles of Tissue Engineering’ (2007) edited by R P Lanza, R Langer, J. Vacanti. Academic Press, London, ISBN: 978-0-12-370615-7.

Suitable polymers include a biodegradable polymer selected from the group consisting of poly(lactic acid), poly(L-lactic acid), poly(D-lactic acid), poly(glycolic acid), polycaprolactone, poly(epsilon-caprolactone), poly(lactide-co-glycolide), poly(epsilon-caprolactone-co-glycolide), poly(epsilon-caprolactone-co-L-lactide), polydioxanone, polygluconate, poly(lactic acid-co-ethylene oxide), polyhydroxybutyrate, poly(hydroxpriopionic acid), polyphosphoester, poly(alpha-hydroxy acid), polycarbonates, polyamides, polyanhydrides, polyamino acids, polyorthoesters, biodegradable polyurethanes, poly(ethyleneoxideterephthalate), poly(butyleneterephthalate), and co-polymers and mixture thereof. In one embodiment, a biodegradable polymer is selected from the group consisting of poly(lactic acid), poly(glycolic acid), poly(epsilon-caprolactone), polydioxanone, poly(lactide-co-glycolide), poly(epsilon-caprolactone-co-glycolide) and poly(epsilon-caprolactone-co-L-lactide).

Oxygen release from a metal peroxide starts upon contact with water, to result in the formation of metal hydroxide and hydrogen peroxide. The metal hydroxide contributes to a (local) increase in pH. Of course, an excessive increase in pH to non-physiological values is undesirable. It was found that the use of a biodegradable polymer which degrades into acid degradation products was at least partially capable of compensating the pH increase due to oxygen release from metal peroxide. Therefore, the use of a polymer which, upon hydrolysis, yields an acid degradation product, such as lactic acid or glycolic acid, is particularly preferred. In a specific aspect, the invention provides a composition comprising poly-(D,L-lactic acid) (PDLLA) or poly-(D,L-lactic-co-glycolic acid) (PLGA), preferably PDLLA, and at least one metal peroxide, preferably CaO₂, MgO₂ or a combination thereof.

Alternatively, or additionally, a composition of the invention may comprise as a further ingredient an agent capable of neutralizing a metal hydroxide into a physiologically acceptable product. Of special interest in this respect is calcium hydrogen phosphate (CaHPO4), in particular when combined with Ca and/or Mg peroxides as oxygen sources. CaHPO4 can remove excess metal hydroxide as follows:

2CaHPO₄+Ca(OH)₂→Ca₃(PO₄)₂+2H₂O

Calcium and magnesium phosphates are natural components of the body and play important roles in the formation and mineralization of bone. Therefore, a composition comprising calcium and/or magnesium peroxide in combination with CaHPO₄ as neutralizing agent is advantageously used in bone tissue engineering.

A composition of the invention may contain one or more further useful additive(s). In one embodiment, it comprises at least one additive that contributes to cell survival, proliferation and/or differentiation. Also encompassed are additives which can neutralize, scavenge or absorb a component that would otherwise affect cell growth or survival in a negative fashion. Exemplary useful additives are nutrients, e.g. fermentable sugars such as glucose, etc., and biologically active agents e.g. cytokines, growth factors, hormones, inflammatory stimuli, angiogenic factors. A number of growth factors exist that are involved in inducing a variety of cellular responses in connection with a variety of cell functions. Some growth factors are for example osteoinductive, whereas other growth factors have inductive effect on articular cartilage.

The growth factor may be selected from the group consisting of platelet derived growth factor (PDGF) AA, PDGF BB, insulin-like growth factors, fibroblast growth factors (FGF), β-endothelial cell growth factor; transforming growth factors (TGF), such as TGF-P1, TGFβ1.2, TGF-β2, TGF-β3, TGF-β5; bone morphogenic protein (BMP) 1, BMP2, BMP 3, BMP 4, BMP 7, vascular endothelial growth factor (VEGF), placenta growth factor; epidermal growth factor (EGF), amphiregulin, betacellulin, heparin binding EGF, interleukins (IL)-1, IL-2, IL-3, IL-4, IL-5, IL-6, IL-7, IL-8, IL-9, IL-10, IL-11, IL-12, IL-13, IL-14, IL-15-18, colony stimulating factor (CSF)-G, CSF-GM, CSF-M, erythropoietin, nerve growth factor (NGF), ciliary neurotropic factor, stem cell factor, and hepatocyte growth factor.

A further aspect relates to an implantable device comprising a composition according to the invention. The implantable device is preferably a scaffold for tissue engineering, more preferably a three-dimensional (3D) scaffold for tissue engineering. Scaffolds for tissue engineering can be designed according to specific needs and requirements using standard technology. Typically, the size and shape of said scaffold is a function of the tissue into which the scaffold is to be implanted. Other important and well known parameters include porosity, mechanical properties, pore size and interconnectivity of the pores. Ideally, the size of the scaffold is easy to adjust, either by cutting with a scalpel or by adding smaller pieces together. Furthermore, the filling of the tissue lesion should in the long term result in a normal tissue architecture. Also, the implanted scaffold should not have adverse effect on the newly generated tissue. It is furthermore desirable that the implant is completely remodelled resulting in a scarless regeneration of the defected tissue.

An implantable device may consist partially or completely of an oxygen-delivering polymer composition described herein above. In one embodiment, the matrix of the device consists essentially of such a composition. For example, it is a scaffold prepared from poly(lactic acid) comprising at least one metal peroxide. In another embodiment, the implantable device has a core or matrix of any suitable material, which core of matrix is coated with a biocompatible, oxygen-delivering composition according to the invention, or vice versa i.e. a core of an oxygen-releasing polymer coated with for instance a calcium phosphate material. In one embodiment, the scaffold is a ceramic scaffold matrix that is coated with a composition as provided herein. The at least one metal peroxide may for instance be used as oxygen source in a coating of poly(lactic-co-glycolic acid) on a hydroxyapatite/tricalcium phosphate (HA/TCP) scaffold (Miao et al. Acta Biomater. 2008 May; 4(3):638-45). The invention also encompasses an injectable oxygen delivering system comprising a polymer composition as described herein above. In one embodiment, microspheres of a metal peroxide/polymer composition are combined with injectable calcium phosphate cement, such as a cement disclosed by Habraken et al. (Biomaterials 29 (2008) 2464-2476).

An example of a suitable scaffold shape is the shape as a sheet which may be suitable for treatment of large dermal defects, fascia defects or other membranes. For example a cylindrical form may be suitable for the repair of focal injuries in articular cartilage. For repair of bone and joint defects the three-dimensional cell scaffold may be in the shape of a sphere. Similarly, a scaffold shape in the form of a rectangle or a cube as well as a sponge-shaped scaffold may be suitable for bone defects. However, the scaffold may be of any irregular shape suitable for a variety tissue defects. The shape of the three-dimensional cell scaffold is not limited to the examples of suitable applications as given above. According to the present invention the shape of the three-dimensional cell scaffold should be suitable for repairing any damage to the tissue as described elsewhere herein.

Generally speaking, the three-dimensional scaffold according to the present invention should be able to accommodate cells that will aid in the repair of the damaged tissue. In order for cells to be cultured inside and on the scaffold the three-dimensional scaffold should comprise cavities suitable in size in which cells should be able to live, multiply, differentiate and form tissue. In the following such cavities will be referred to as pores. The cell scaffold therefore typically has pores. The presence of pores may also allow for the population of the scaffold by cells originating from surrounding tissues by invasion. Thus, in general the size of the pores will range from about one to ten times the diameter of the cells to be seeded in the scaffold. The size of the pores is thus adapted to the type cell to be accommodated within the three dimensional scaffold considering which type of tissue is to be regenerated or repaired. It is important for the pores to be of a sufficiently large size (sufficient pore volume) so as to allow cells (i.e., living cells) to maintain their shape within the structure. Furthermore, a large pore volume is desirable in order to allow a cell suspension to fully penetrate the structure and thus permit cell seeding and/or cell migration throughout the material. Also, the pores should be interconnected and the interconnections should be of sufficient size. In relation to access to nutrients and efficient removal of waste products following cellular metabolism a sufficient pore volume is needed. According to the present invention the pores of the cell scaffold have a pore size in the range of from about μm to 1000 μm, such as 50 μm to 1000 μm, for example 100 μm to 900 μm, such as 200 μm to 600 μm.

The pores of the three-dimensional cell scaffold should be relatively uniform in size, which ensures that the pores are large enough to accommodate the living cells in a uniform manner throughout the three-dimensional scaffold. Thus, in one embodiment of the invention the pores of three-dimensional cell scaffold are uniform in size.

The scaffold may further comprise living cells. Once the cells are inoculated on and/or inside the scaffold the cells will proliferate on and/or inside the scaffold. Thus, the three-dimensional scaffold comprising cells can be used in vivo. The three-dimensional cell scaffold according to the present invention will sustain active proliferation of the culture for long periods of time or the desired period of time. The cells employed in the present invention may be characterized by different donor-recipient relationships. The term ‘xenogeneic’ means cells or tissue from donor individuals belonging to a different species than the recipient individual. One example is a graft of cells or tissues from a pig which is transplanted or transferred according to the present invention to a human. The term ‘allogeneic’ refers to cells or tissue from individuals belonging to the same species but from genetically different individuals. The term ‘syngeneic’ refers to cells or tissue from a donor individual who is genetically identical to the recipient individual, for example where donor and recipient are identical twins belonging to different species. The term ‘autologous’ refers to cells or tissue from the same individual, for example where cells are from the patient him- or herself. Thus, according to the present invention the three-dimensional scaffold may further comprise cells selected from the group consisting of autologous, xenogeneic, allogeneic and syngeneic cells. In one embodiment the cells may be syngeneic cells in order to avoid rejection of cells due to incompatibility, immunological rejection of the transplant and/or graft versus host disease is likely. Another embodiment is the use of xenogeneic cells, or for example allogeneic cells. In a preferred embodiment the cells are autologous whereby rejection of cells due to incompatibility is avoided.

According to the present invention also genetically modified cells may be used, which have been created to be particularly useful for the regeneration of tissue, such as bone, tendon, ligament and/or cartilage. The cells may be genetically engineered to produce gene products beneficial to transplantation, e.g. anti-inflammatory factors, e.g., anti-GM-CSF, anti-TNF, anti-IL-1, anti-IL-2, etc. Alternatively, the cells may be genetically engineered to “knock out” expression of native gene products that promote inflammation, e.g., GM-CSF, TNFα, IL-1, IL-2, or “knock out” expression of MHC in order to lower the risk of rejection. In addition, the cells may be genetically engineered for use in gene therapy to adjust the level of gene activity in a patient to assist or improve the results of the cartilage transplantation by use of the three-dimensional cell scaffold according to the present invention. When cells are obtained from a donor or a patient, the cells can be obtained by small biopsies for example 5 mg to 1000 mg of tissue from a patient and/or a donor, such as 50 mg to 1000 mg, for example 100 mg to 1000 mg, such as 200 mg to 1000 mg, for example 300 to 1000 mg, such as 400 mg to 1000 mg, for example 500 mg to 1000 mg. The quantity required for treatment of a tissue defect depends on the volume of the tissue defect, the cellular density in the recipient tissue, and/or the proliferal potential of the donor cells.

Accordingly, also provided is an implantable device comprising living cells, preferably mammalian cells, most preferably human cells. Depending on the desired application or tissue lesion to be treated, any desired cell type or mixture of two or more cell types can be seeded onto or into the device. Examples include bone marrow cells, cartilage cells, osteoblasts, mesenchymal stem cells, embryonic stem cells, gene transfected cells, subcutaneous fat derived mesenchymal stem cells, endothelial cells and combinations thereof.

In the case of cartilage cells, these may be obtained from any cartilage source in the body, including any articular surface e.g. knee joint, ankle joint, shoulder joint etc.; or from the costal cartilage, rib cartilage, nose cartilage, ear cartilage, symphysis, intervertebral discs, meniscus, soft palate, laryngeal cartilage or tracheal cartilage. When removing cartilage, bone or muscle cells from the patient or donor, this is advantageously done using an air-tight operation method avoiding donor cell exposure to atmospheric air. By preventing exposure to atmospheric air the cells will produce implants of higher quality (e.g. cartilage implants with a higher content of type II collagen) than if the cells are exposed to ambient oxygen.

In accordance with the invention, cells can be inoculated onto and/or inside the three-dimensional cell scaffold, and grown in culture to form a living tissue material. The cells may be fetal or adult in origin, and may be derived from convenient sources such as cartilage, skin or bone. Such tissues and/or organs can be obtained by appropriate biopsy or upon autopsy; cadaver organs may be used to provide a generous supply of stromal cells and elements. Alternatively, umbilical cord and placenta tissue or umbilical cord blood may serve as an advantageous source of fetal-type cells, e.g., osteoblast-progenitors, chondrocyte-progenitors and/or fibroblast-like cells for use in the three-dimensional cell scaffold of the invention.

The proliferation step, which may precede the inoculation of the three-dimensional cell scaffold may serve two purposes. First and foremost, the cells are proliferated to simply increase their number. In addition to this, cells may also—at least partly—de-differentiate during the proliferation step. In the case of stem cells, differentiation into muscle, bone and/or chondrocyte cells may take place during the proliferation step. It is convenient that the proliferation is performed in flasks, such as culture flasks, or in Petri dishes, or for example rolling flasks, or three-dimensional culture systems. MSC are for example proliferated in monolayer culture or in spinner flasks on macroporous microcarriers (Malda et al. Biomat (2003) 24(28): 5133-5161). Chondrocytes may be proliferated initially in monolayer for one to two cell divisions and after that transferred and proliferated under growth conditions allowing three-dimensional growth.

The present invention also relates to a method for producing a three dimensional scaffold for tissue engineering, comprising providing a mold of a size and shape approximating the tissue into which said scaffold is to be implanted; providing a solution of at least one synthetic biocompatible polymer and at least one peroxide in an organic solvent, such as ethyl acetate or for example chloroform, and forming the scaffold by solvent casting. The procedure of solvent casting is well established in the art of TE scaffolds. A three-dimensional cell scaffold formed by the disclosed steps is also within the scope of the present invention. Similarly, a method for producing a ready-for use implant as describe herein is also within the scope of the present invention. The method may further comprising the step of applying living cells to said scaffold.

Still a further aspect relates to a method of forming tissue (in vitro or in vivo), the method comprising (a) providing an implantable device according to the invention, (b) covering at least part of the surface of the scaffold with living cells capable of forming tissue; and (c) culturing the scaffold under conditions suitable to grow tissue on and/or in the scaffold.

Of course, another aspect relates to the therapeutic uses of an implantable device of the invention. In one embodiment there is provided a method for regenerating tissue in a mammal in need thereof, comprising implanting the three-dimensional cell scaffold of the present invention. In another embodiment, the invention provided a method for regenerating tissue in a mammal in need thereof, comprising implanting the three-dimensional cell scaffold of the present invention. The mammal is for example a goat, mouse, rabbit, rat, pig, dog, horse, cat, cow or a human. In a preferred embodiment the mammal is a human.

Also encompassed is a method for treating a tissue pathology in a subject, the method comprising (a) providing an implantable device according to the invention, (b) covering at least part of the surface of the scaffold with living cells capable of forming tissue; and (c) culturing the scaffold under conditions suitable to grow tissue on and/or in the scaffold, and (d) introducing the scaffold into the subject, and wherein the cells used in step (b) are capable of treating the tissue pathology. The tissue pathology for instance comprises loss, damage, injury, or combinations thereof to the tissue. The treatment comprises for example tissue remodeling, repair, regrowth, resurfacing, regeneration, or combinations thereof.

The tissue according to the present invention may be bone, cartilage, tendon, ligament, nerve, skin, vascular, cardiac, pericardial, muscle, ocular, periodontal, breast, pancreatic, esophageal, stomach, kidney, hepatic, mammary, adrenal, urological, and intestinal tissue. Thus, in one embodiment of the invention the tissue that is to be regenerated or treated may be selected form the group consisting of bone, cartilage, tendon, ligament, nerve, skin, vascular, cardiac, pericardial, muscle, ocular, periodontal, breast, pancreatic, esophageal, stomach, kidney, hepatic, mammary, adrenal, urological, and intestinal tissue. In another embodiment the tissue may be selected form the group consisting of bone, cartilage, tendon and ligament. In a particular embodiment the tissue is cartilage. Alternatively, the tissue is bone.

Mammals in need of regenerating tissue comprise mammals wherein injury to tissue has occurred. Surgical intervention is often required to repair the damage. Such surgical repairs can include suturing or otherwise repairing the damaged tissue with known medical devices, augmenting the damaged tissue with other tissue, using an implant, a graft or any combination of these techniques. According to the present invention tissue may be regenerated in a mammal by implanting the three-dimensional scaffold of the present invention. In one embodiment of the invention the mammal may be suffering from a defect to tissue selected from the group consisting of articular cartilage defects, meniscal defects, discus intervertebralis defects, bone defects, vertebral body fractures, skin wounds, fascial defects, tendon ruptures, ligament ruptures, nerve injuries, spinal cord injuries, blood vessel defects, ear substitution, nasal cartilage defects, muscle defects, heart muscle defects, muscle degeneration, adipose defects, tooth injuries, bladder wall defects, gastric wall defects, intestinal wall defects, pancreatic island transplantation, and eye injuries.

The invention is exemplified by the Examples below.

### LEGENDS TO THE FIGURES

FIG. 1. pH of PBS in which 5 different scaffolding materials were incubated.

FIG. 2. Reduction in weight of the materials in 20 days.

FIG. 3: Oxygen release in CaO₂- and MgO₂-containing materials for 24 hours.

FIG. 4: Change of pH of PBS in CaO₂- and MgO₂-containing materials in 24 hours.

FIG. 5: Oxygen release from materials prepared from compositions comprising different amounts of CaO₂ and MgO₂. In the third material CaHPO₄ was added to neutralize hydroxide.

FIG. 6: Change of pH due to the presence of peroxide containing materials in 24 hours. Addition of CaHPO₄ to the material inhibits the increase of pH.

FIG. 7: Oxygen release and pH change by PCM90:5:5 during 26 days. After each assessment the PBS was refreshed.

FIG. 8: Viability of cells seeded on different materials after 24 hours. The composition of the materials in indicated on the photographs.

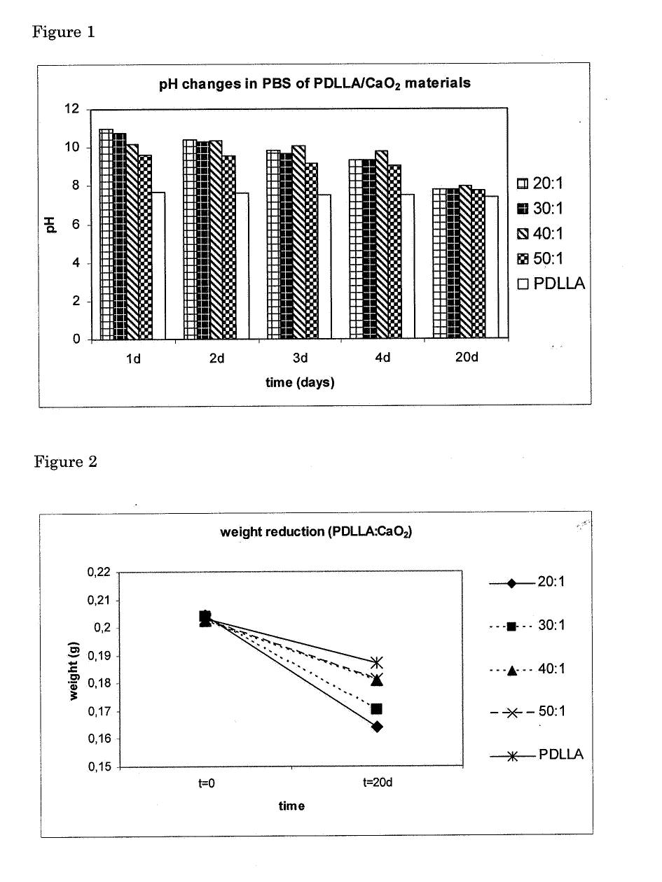

### EXPERIMENTAL SECTION

### Materials and Methods

PDLLA was a medical grade material obtained from Purac, Weesp, The Netherlands. CaO₂, MgO₂ and CaHPO₄ were obtained from Sigma-Aldrich.

PDLLA was dissolved in chloroform in a concentration of 1 g/10 ml. To this solution an appropriate amount of metal peroxide, and optionally CaHPO4, was added (see individual experiments). The solution was thoroughly stirred and eventually the solution was briefly sonicated in a sonication bath. Several compositions were prepared in this fashion, each comprising different amounts of polymer, metal oxide(s), and neutralizing agent (see Table 1).

Then, films were cast of the compositions. The films were allowed to dry and dried further under vacuum.

TABLE 1
Compositions produced and tested.

| Film | PDLLA | CaO₂ | MgO₂ | CaHPO₄ |
|---|---|---|---|---|
| P | 100 | | | |
| PC1:1 | 50 | 50 | | |
| PC5:1 | 80 | 20 | | |
| PC10:1 | 90 | 10 | | |
| PC20:1 | 95 | 5 | | |
| PC30:1 | 96.7 | 3.3 | | |
| PC40:1 | 97.5 | 2.5 | | |
| PC50:1 | 88 | 2 | 10 | |
| PM1:1 | 50 | | 50 | |
| PM1:5 | 80 | | 20 | |
| PM1:10 | 90 | | 10 | |
| PCM90:5:5 | 90 | 5 | 5 | |
| PCM85:10:5 | 85 | 10 | 5 | |
| PCM85:5:10 | 85 | 10 | 5 | |
| PCMD85:5:5:5 | 85 | 5 | 5 | 5 |
| PCMD80:5:10:5 | 80 | 5 | 10 | 5 |
| PCM80:5:15 | 80 | 5 | 15 | |

Values indicate the relative amounts of the components, expressed as weight percent of the total dry weight of the composition. P indicates polymer (PDLLA); C denotes calcium peroxide; M denotes magnesium peroxide and D denotes CaHPO₄. As used herein, the annotation “PC1:1” refers to a film prepared from a mixture in polymer and calcium peroxide in a 1:1 weight ratio. Likewise, PM1:10 refers to a film prepared from a mixture in polymer and magnesium peroxide in a 1:10 weight ratio, and so on.

### Experiment 1

We assessed the increase in pH due to the formation of metal hydroxide concentration when a metal peroxide reacts with water. Apiece of approximately 2 cm² casted film prepared from 5 different compositions (PDLLA alone, and PDLLA plus CaO₂ in a relative weight ratio of 20:1; 30:1; 40:1 and 50:1) was put in phosphate-buffered saline (PBS: 0.01 M phosphate buffer containing 0.14 M NaCl) (pH=7.6). The pH was measured every day and the PBS was not refreshed. The pH of the buffer comprising a piece of PDLLA without metal peroxide was about 7.8, and was stable up to at least 20 days. Films prepared from PDLLA comprising metal peroxides caused an initial increase in pH to a value ranging from about 9.5 to about 11, although the pH decreased in time to values similar to PDLLA alone after 20 days. Most probably, an increased degradation due to a high pH results in the production of lactic acid, which will then lower the pH.

FIG. 2 shows the kinetics of weight reduction of the different materials. The rate of weight reduction of material comprising metal peroxides is higher than that of PDLLA alone, and is likely to be caused by an increase in the degradation of PDLLA by increased pH.

### Experiment 2

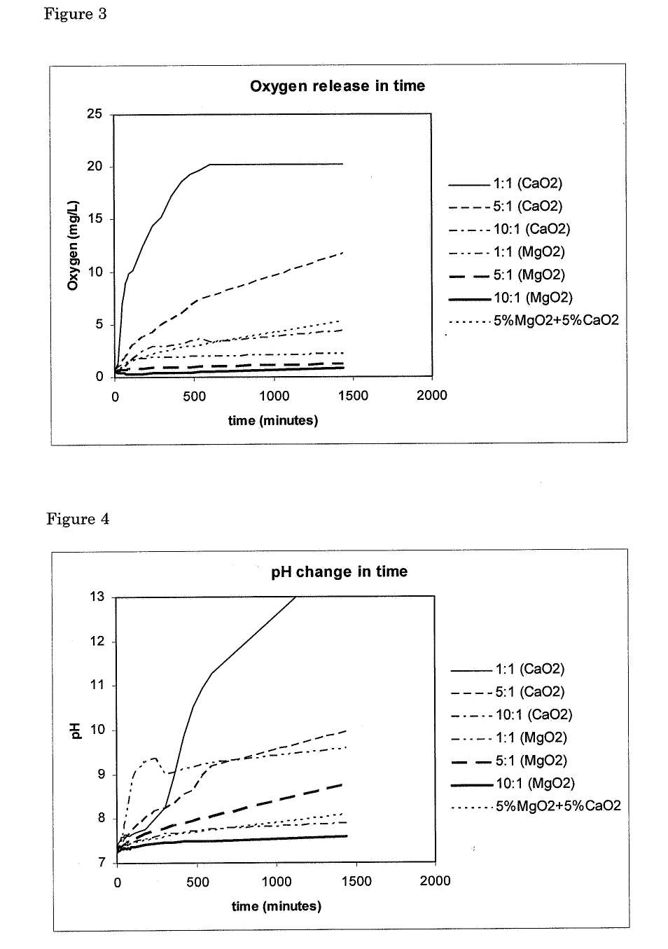

Approximately 2 cm² of material was placed in deoxygenized PBS and oxygen concentrations were monitored for 24 hours. The materials tested were prepared from the following compositions (see table 1): PC1:1; PC5:1; PC10:1; PM1:1; PM5:1; PM10:1 and PCM90:5:5. The results are shown in FIG. 3. Materials comprising only CaO₂ show a rapid and strong release of oxygen, whereas MgO₂-containing materials exhibit a slow yet sustained oxygen release profile. A combination of both metal peroxides yields a release profile with attractive characteristics in terms of the amount and the kinetics of oxygen production.

FIG. 4 depicts the change in pH of PBS upon incubation of the materials. Some materials caused an increase in pH above physiologically acceptable levels. We decided to aim at a pH as close as possible to the physiologic pH and set an upper limit of 7.8-8.0. Of the materials tested, PM90:10, PC90:10 and PCM90:5:5 fulfilled this requirement.

### Experiment 3

This is a similar experiment as the previous one, with freshly prepared materials.

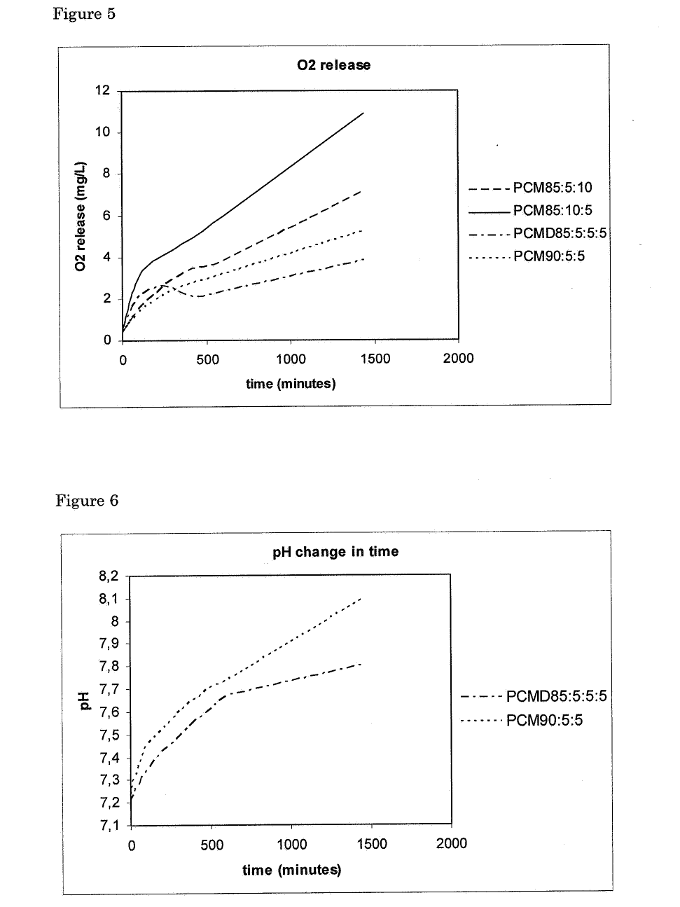

FIG. 5 shows the oxygen release profiles from materials prepared from compositions comprising different amounts of CaO₂ and MgO₂. In the third material CaHPO4 was added to neutralize metal hydroxide.

FIG. 6 shows the change of pH of a PBS buffer over a period of 24 hours due to the presence of peroxide containing materials. Incorporation of CaHPO₄ in the material inhibits the increase of pH.

Based on these data we decided to test these materials for a longer period.

### Experiment 4

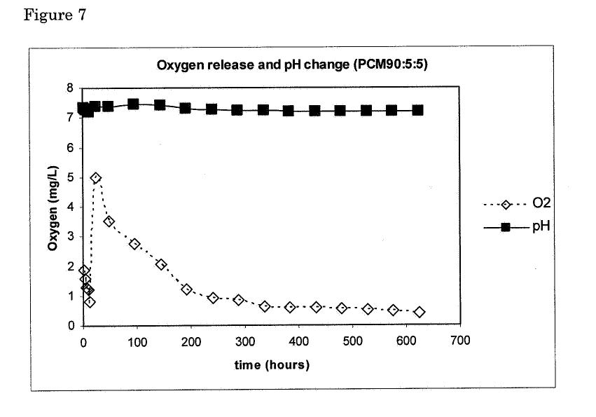

A composition was prepared from PDLLA, CaO₂ and MgO₂ (90:5:5 by weight) and a film was cast. Oxygen release and pH were monitored up to 26 days. The results are shown in FIG. 7. It was found that the film releases 0.4 mg/L oxygen for after 26 days. This is equivalent with 12.5 nmol/ml. The release is the highest in the first 24 hours and then gradually decreases. The high release is caused by the CaO₂. Oxygen release because of the reaction of MgO₂ is a much slower process.

### Experiment 5

### Biocompatibility of the Materials

The next question was whether cells would survive on the peroxide containing materials. For this purpose, films were prepared of different compositions (PCM90:5:5; PCMD85:5:5:5; PM90:10; PMD85:10:5 and PDLL alone) and sterilized those with UV light and 70% ethanol.

In first instance we tested this in normoxic conditions using murine MC3T3-E1 osteoblast-like cells. The cells were harvested from tissue culture flasks using trypsin-EDTA treatment (standard cell culture procedure). Then the cells were suspended in culture medium and a drop of the suspension was put on top of a film. Cells were allowed to sediment and attach, and after 2-3 hours culture medium was added. At different moments up to 24 hours, a film was taken and incubated with culture medium containing 3-(4,5-dimethylthiazol-2-yl)-2,5-diphenyl tetrazolium bromide (MTT). MTT is water-soluble yellowish substance, which is converted in mitochondria of viable cells into a purple-black insoluble precipitate, formazan. After approximately 3-4 hours the reaction is complete and photographs were taken.

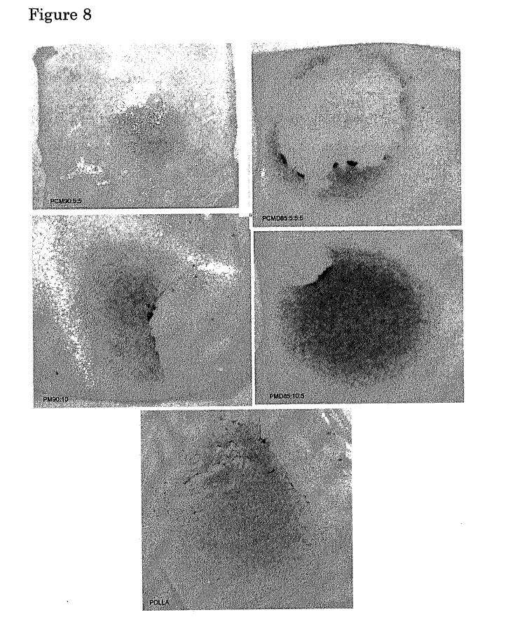

FIG. 8 shows the viability of cells on different materials. The composition of the materials in indicated on the photographs. Unfortunately, majority of the cells were lost from the films PCMD85:5:5:5 en PM90:10 during processing. From FIG. 8 it was concluded that all tested materials appear to be biocompatible. Especially materials prepared from PCMD85:5:5:5 and PMD85:10:5 show excellent cell viability.

The above data demonstrate that a composition comprising a biocompatible polymer (PDLLA) and CaO₂ and/or MgO₂ and casted as a film releases oxygen for a considerable number of days. Furthermore, the pH change due to the concomitant produced hydroxide is not of any major consequence at low concentrations of peroxides, and can be controlled using CaHPO₄. The resulting Ca₁₀(PO₄)₆(OH)₂ is a physiological product in bone. The materials are all biocompatible for at least 24 hours.

1. A composition comprising (i) at least one biocompatible polymer suitable for use in tissue-engineering scaffolds and (ii) at least one metal peroxide.

2. Composition according to claim 1, wherein the polymer is present in an amount of at least 60 weight %, preferably at least 70 weight %, more preferably at least 80 weight % based on the total dry weight of the composition.

3. Composition according to claim 1, comprising at least 2 weight %, preferably at least 5 weight %, more preferably at least 10 weight % of metal peroxide based on the total dry weight of the composition.

4. Composition according to claim 1, wherein said metal peroxide is CaO₂, MgO₂, or a combination thereof.

5. Composition according to claim 4, comprising CaO₂ and MgO₂, preferably in a relative amount of between 10:1 and 1:10 by weight, more preferably between 5:1 and 1:5.

6. Composition according to claim 1, wherein said biocompatible polymer is a biodegradable polymer, preferably a biodegradable synthetic polymer.

7. Composition according to claim 6, wherein said biodegradable polymer is selected from the group consisting of poly(lactic acid), poly(L-lactic acid), poly(D-lactic acid), poly(glycolic acid), polycaprolactone, poly(epsilon-caprolactone), poly(lactide-co-glycolide), poly(epsilon-caprolactone-co-glycolide), poly(epsilon-caprolactone-co-L-lactide), polydioxanone, polygluconate, poly(lactic acid-co-ethylene oxide), polyhydroxybutyrate, poly(hydroxpriopionic acid), polyphosphoester, poly(alpha-hydroxy acid), polycarbonates, polyamides, polyanhydrides, polyamino acids, polyorthoesters and biodegradable polyurethanes.

8. Composition according to claim 7, wherein said biodegradable polymer is selected from the group consisting of poly(lactic acid), poly(glycolic acid), poly(epsilon-caprolactone), polydioxanone, poly(lactide-co-glycolide), poly(epsilon-caprolactone-co-glycolide) and poly(epsilon-caprolactone-co-L-lactide).

9. Composition according to claim 1, further comprising (iii) at least one substance capable of neutralizing metal hydroxides into a physiologically acceptable product.

10. Composition according to claim 9, wherein said neutralizing substance is CaHPO₄.

11. Composition according to claim 1, further comprising (iv) at least one additive that contributes to cell survival, proliferation and/or differentiation, preferably wherein said at least one additive is a nutrient or a biologically active agent.

12. An implantable device comprising a composition according to claim 1.

13. Implantable device according to claim 12, being a scaffold for tissue engineering.

14. Implantable device according to claim 13, wherein the scaffold matrix essentially consists of a composition comprising (i) at least one biocompatible polymer suitable for use in tissue-engineering scaffolds and (ii) at least one metal peroxide.

15. Implantable device according to claim 13, wherein the scaffold is a ceramic scaffold matrix that is coated with a composition comprising (i) at least one biocompatible polymer suitable for use in tissue-engineering scaffolds and (ii) at least one metal peroxide.

16. Implantable device according to claim 1, the device being provided with living cells, preferably mammalian cells, most preferably human cells.

17. Implantable device according to claim 16, provided with bone marrow cells, osteoblasts, mesenchymal stem cells, cartilage cells, embryonic stem cells, gene transfected cells, endothelial cells and combinations thereof.

18. The use of a peroxide, preferably a metal peroxide, as an in situ oxygen-delivering substance in tissue engineering.

19. A method of forming tissue, the method comprising (a) providing an implantable device according to claim 12, (b) covering at least part of the surface of the scaffold with living cells capable of forming tissue; and (c) culturing the scaffold under conditions suitable to grow tissue on and/or in the scaffold.

20. A method for the treatment of a tissue pathology in a subject, the method comprising steps (a), (b) and (c) of claim 19, followed by step (d) of introducing the scaffold into the subject, and wherein the cells used in step (b) are capable of treating the tissue pathology.

21. Method according to claim 20, wherein the tissue pathology comprises loss, damage, injury, or combinations thereof to the tissue.

22. Method according to claim 20, wherein the treatment comprises tissue remodeling, repair, regrowth, resurfacing, regeneration, or combinations thereof.

23. A method for providing a scaffold for tissue engineering, comprising:
providing a mold of a size and shape approximating the tissue into which said scaffold is to be implanted;
providing a solution of at least one synthetic biocompatible polymer and at least one peroxide in an organic solvent, and
forming the scaffold by solvent casting.

24. The method according to claim 23, further comprising the step of applying living cells to said scaffold.
